(19) United States
(12) Patent Application Publication
(10) Pub. No.: US 2025/0339645 A1
(43) Pub. Date: Nov. 6, 2025

(54) Deflectable Catheter Distal End

(71) Applicant: St. Jude Medical, Cardiology Division, Inc., Saint Paul, MN (US)

(72) Inventors: Troy Tegg, Elk River, MN (US); Greg Olson, Elk River, MN (US); Tim Marass, Minneapolis, MN (US); Fermin Lupotti, Lake Forest, CA (US); Xuan Khieu, Maple Grove, MN (US)

(73) Assignee: St. Jude Medical, Cardiology Division, Inc., Saint Paul, MN (US)

(21) Appl. No.: 18/854,188

(22) PCT Filed: Mar. 28, 2023
(86) PCT No.: PCT/US2023/016596

§ 371 Date: Oct. 4, 2024

Related U.S. Application Data
(60) Provisional application No. 63/329,244, filed on Apr. 8, 2022.

(51) Int. Cl. A61M 25/01; A61M 25/00

(52) U.S. Cl. CPC A61M 25/0144; A61M 25/0026; A61M 25/0043; A61M 25/0147; A61M 2025/0036

(57) ABSTRACT

Described is a steerable catheter assembly including an elongated catheter shaft with a steerable section that can be articulated to navigate the catheter shaft through a tortuous path through a patient's vasculature. The steerable section includes a planarity member(s) disposed within and fixedly attached (e.g., row of holes extending along a length of the planarity member). The planarity member extend along the centerline of the steerable section. The planarity member has a cross-sectional area perpendicular to the centerline. The cross-sectional area has a maximum principal area moment of inertia of at least 5 times a minimum principal area moment of inertia. The catheter shaft also includes a deflection lumen offset from the planarity member, and a pull wire disposed within the deflection lumen and operable to induce deflection of the elongated catheter shaft section.

### CROSS REFERENCE TO RELATED APPLICATIONS

The present application claims the benefit of U.S. Provisional Application No. 63/329,244 filed Apr. 8, 2022; the full disclosure which is incorporated herein by reference in its entirety for all purposes.

### FIELD OF DISCLOSURE

This disclosure relates generally to a deflectable catheter and related components. More particularly, this disclosure relates to deflectable portions of deflectable catheters.

### BACKGROUND

Medical devices, catheters, and/or cardiovascular catheters, such as electrophysiology catheters, can be used in a variety of diagnostic, therapeutic, and/or mapping and ablative procedures to diagnose and/or correct conditions such as atrial arrhythmias, including for example, ectopic atrial tachycardia, atrial fibrillation, and atrial flutter. Arrhythmias can produce a variety of medical conditions including irregular heart rates, loss of synchronous atrioventricular contractions, and stasis of blood flow in a chamber of a heart, which can lead to a variety of other symptomatic and asymptomatic ailments and even death.

Ablation therapy can be used to treat various medical conditions. One medical condition in which ablation therapy may be used is the treatment of cardiac arrhythmias. It is believed that the primary cause of atrial arrhythmia is stray electrical signals within the left or right atrium of the heart. An ablation catheter can be used to impart ablative energy (e.g., radiofrequency energy, electroporation, cryoablation, lasers, chemicals, high-intensity focused ultrasound, etc.) to cardiac tissue to create a lesion in the cardiac tissue that disrupts undesirable electrical pathways and thereby limits or prevents stray electrical signals that lead to arrhythmias.

Electroporation is a non-thermal ablation technique in which an electric field is applied to tissue to induce pore formation in cellular membranes. The electric field can be applied in a pulse train of relatively short duration pulses that last, for example, from a nanosecond to several milliseconds. When electroporation is applied to tissue in an in vivo setting, the cells in the tissue are subjected to a trans-membrane potential to induce the pore formation in the cellular membranes. Electroporation may be reversible (i.e., the induced pores are temporarily formed) or irreversible (i.e., the induced pores remain open and induce cellular destruction). In the field of gene therapy, reversible electroporation is used to accommodate transportation of high molecular weight therapeutic vectors into cells. In other therapeutic applications, irreversible electroporation is used to induce cell destruction.

A catheter can include a steerable section that can be selectively articulated to enhance advancement of the catheter along a path (which is often tortuous) though the patient's vasculature. Typically, the steerable section is located near the distal end of the catheter and one or more pull wires are employed to transmit steering forces from a proximal handle assembly to the steerable section. Examples of catheters with steerable sections are disclosed in U.S. Pat. Nos. 4,817,613, and 7,914,515, which are incorporated herein in its entirety by reference.

### BRIEF SUMMARY

The present disclosure relates to steerable catheter assemblies, including bi-directional catheters, used during medical procedures such as, for example, diagnostic, therapeutic, and/or mapping and ablative procedures to diagnose and/or correct conditions such as atrial arrhythmias (e.g., ectopic atrial tachycardia, atrial fibrillation, and atrial flutter). In many embodiments, a steerable catheter assembly includes an elongated catheter shaft with a steerable section that can be articulated to navigate the catheter shaft through a tortuous path through a patient's vasculature. The steerable catheter can include one or more lumens for passage of electrical wires, fluid, or other therapeutic or diagnostic elements (e.g., sensors). In many embodiments, the steerable section is actuatable to selectively induce a desired amount of curvature into the steerable section while maintaining planarity of the steerable section, which enhances the ability to navigate the catheter shaft through the patient's vasculature. In many embodiments, the steerable section includes one or more large diameter lumens relative to the outside diameter of the steerable section (which is typically limited in size to accommodate usage within a patient's vasculature) so to accommodate the usage of the catheter in some medical procedures that require one or more large diameter lumens.

Thus, in one aspect, a steerable catheter includes the elongated catheter shaft section having a distal end, a proximal end, and a centerline that extends between the distal end and the proximal end. The steerable section is formed towards the distal end of the elongated catheter shaft section. To articulate the steerable section, a pull wire is disposed within a deflection lumen and is actuatable to induce deflection of the steerable section. The deflection lumen is eccentrically located, e.g., offset from a centerline of the steerable section. In many embodiments, the steerable section includes two offset deflection lumens with each of the deflection lumens housing a respective pull wire that is actuatable to induce deflection in the steerable section in a respective direction so that deflection of in the steerable section can be induced in two different directions.

The steerable section includes a planarity member. The planarity member is disposed within and attached to the steerable section and extends along the steerable section. In many embodiments, the planarity member is fixedly attached to the steerable section via a molded member that is interfaced with the planarity member. In some embodiments, the planarity member includes one or more rows of holes to enhance coupling of the planarity member with the molded member. The planarity member has a cross-sectional area perpendicular to the centerline. The cross-sectional area has a minimum principal area moment of inertia and a maximum principal area moment of inertia. The maximum principal area moment of inertia is at least 5 times the minimum principal area moment of inertia. In many embodiments, the planarity member has an elongated rectangular cross-section having a width in a range of 0.025 to 0.120 inches and a thickness in a range of 0.002 to 0.008 inches. In many embodiments, the planarity member has a higher elastic modulus than the molded member or the elongated catheter shaft section.

The planarity member serves to constrain deflection of the steerable section to inhibit out of plane deflection of the steerable section. Additionally, the planarity member serves to increase the axial stiffness of the steerable section to enhance the ability to distally advance the steerable section through a patient's vasculature. In many embodiments, the planarity member has a low torsional stiffness to inhibit storage of torsional moment within the steerable section so that undesired torsional movement of the steerable section within the patient's vasculature can be inhibited. In many embodiments, the planarity member is thin as compared to a cylindrical stiffness member and therefore leaves more room withing the steerable section for one or more large diameter lumens (e.g., 13 French), and/or additional lumens compared to existing catheters while providing improved deflection control in a desired deflection plane.

According to another aspect, a bi-directional steerable catheter assembly is provided. The bi-directional steerable catheter assembly includes an elongated catheter shaft section having a distal end, a proximal end, and a centerline that extends between the distal end and the proximal end, at least one planarity member disposed within and attached to the elongated catheter shaft section and extending along the centerline, wherein the at least one planarity member has a substantially rectangular cross-sectional area perpendicular to the centerline and at least one row of holes extending along a length of the planarity member and fixedly attaching the planarity member to the elongated catheter shaft section. The bi-directional steerable catheter assembly also includes a first deflection lumen offset from the at least one planarity member, a second deflection lumen offset from the at least one planarity member, a first pull wire disposed within the first deflection lumen and operable to induce deflection of the elongated catheter shaft section in a first direction, and a second pull wire disposed within the second deflection lumen and operable to induce deflection of the elongated catheter shaft section in a second direction.

A material of the at least one planarity member has a higher durometer than a material of the elongated catheter shaft section. The at least one planarity member has a width in a range of 0.025 to 0.120 inches and a thickness in a range of 0.002 to 0.008 inches. The at least one planarity member has a length in a range of 2 inches to 4 inches. In an example, the at least one planarity member is a single member having three rows of holes extending along a length of the planarity member.

In the bi-directional steerable catheter assembly, the second deflection lumen is disposed on an opposite side of the at least one planarity member relative to the first deflection lumen.

The bi-directional steerable catheter assembly further includes a third lumen disposed on a first side of the at least one planarity member, and a fourth lumen disposed on a second side of the at least one planarity member opposite to the first side. Each of the third lumen and the fourth lumen has a cross-sectional area that is greater than a cross-sectional area of each of the first deflection lumen and the second deflection lumen.

The bi-directional steerable catheter assembly further includes a fifth lumen and a sixth lumen, each of the fifth lumen and the sixth lumen is offset from the centerline of the elongated catheter shaft section.

According to yet another aspect, another bi-directional steerable catheter assembly is provided. The bi-directional steerable catheter assembly includes an elongated catheter shaft section having a distal end, a proximal end, and a centerline that extends between the distal end and the proximal end, at least one planarity member disposed within and attached to the elongated catheter shaft section and extending along the centerline, wherein the at least one planarity member has a solid cross-sectional area perpendicular to the centerline and without a close cell. The cross-sectional area has a width to thickness ratio of at least 5 to 1.

The bi-directional steerable catheter assembly includes a first deflection lumen offset from the at least one planarity member, a second deflection lumen offset from the at least one planarity member; and a first pull wire disposed within the first deflection lumen and operable to induce deflection of the elongated catheter shaft section in a first direction, and a second pull wire disposed within the second deflection lumen and operable to induce deflection of the elongated catheter shaft section in a second direction.

The at least one planarity member has a width in a range of 0.025 to 0.120 inches and a thickness in a range of 0.002 to 0.006 inches. The at least one planarity member has a length in a range of 2 inches to 4 inches. The cross-sectional area of the at least one planarity member has a width to thickness ratio of at least 10 to 1. The at least one planarity member has a rectangular cross-section perpendicular to the centerline.

In the bi-directional steerable catheter assembly the second deflection lumen is disposed on an opposite side of the at least one planarity member relative to the first deflection lumen.

The bi-directional steerable catheter assembly further includes a third lumen disposed on a first side of the at least one planarity member, and a fourth lumen disposed on a second side of the at least one planarity member opposite to the first side. Each of the third lumen and the fourth lumen has a cross-sectional area that is greater than a cross-sectional area of each of the first deflection lumen and the second deflection lumen.

The bi-directional steerable catheter assembly further includes a fifth lumen and a sixth lumen. Each of the fifth lumen and the sixth lumen is offset from the centerline of the elongated catheter shaft section.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and can or cannot represent actual or preferred values or dimensions. Where applicable, some or all features cannot be illustrated to assist in the description of underlying features.

FIG. 1 illustrates a steerable catheter, in accordance with some embodiments of the present disclosure.

FIG. 2 illustrates a steerable section of the steerable catheter of FIG. 1.

FIG. 3 is a cross-section view of an embodiment of the steerable section of FIG. 2 that includes two planarity members.

FIG. 4 illustrates the steerable section of FIG. 3.

FIG. 5 is a cross-sectional view of an embodiment of the steerable section of FIG. 2 that includes one planarity member.

FIG. 6 is a cross-sectional view of another embodiment of the steerable section of FIG. 2 that includes one planarity member.

FIG. 7 and FIG. 8 are longitudinal cross-sectional views of the embodiment of the steerable section of FIG. 3.

FIG. 9 illustrates a planarity member with one row of holes that can be included in the steerable section of FIG. 2.

FIG. 10 illustrates a planarity member with two rows of holes that can be included in the steerable section of FIG. 2.

FIG. 11 illustrates a planarity member with three rows of holes that can be included in the steerable section of FIG. 2.

FIG. 12 illustrates stiffness reference planes for the steerable section of FIG. 2.

FIG. 13 illustrates the orientation of a deflection plane relative to a bending moment applied to the steerable section of FIG. 2.

FIG. 14 illustrates in-plane deflection of the steerable section of FIG. 2.

FIG. 15, FIG. 16, and FIG. 17 are cross-section views of additional embodiments of the steerable section of FIG. 2.

FIG. 18 is a schematic and block diagram of a system for electroporation therapy that can include the steerable catheter of FIG. 1.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s).

However, it will be apparent to those skilled in the art that the disclosed embodiment(s) can be practiced without those specific details. In some instances, well-known structures and components can be shown in block diagram form in order to avoid obscuring the concepts of the disclosed subject matter.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics can be combined in any suitable manner in one or more embodiments. Further, it is intended that embodiments of the disclosed subject matter cover modifications and variations thereof.

It is to be understood that terms such as “distal,” “proximal,” “top,” “bottom,” “front,” “side,” “length,” “inner,” and the like that can be used herein merely describe points of reference and do not necessarily limit embodiments of the present disclosure to any particular orientation or configuration. As used herein, “proximal” refers to a direction toward the end of the catheter near the clinician and “distal” refers to a direction away from the clinician and (generally) inside the body of a patient. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

The terms “longitudinal,” “axial” or “axially” are generally longitudinal as used herein to describe the relative position related to a catheter, a catheter handle, or other components of the system herein. For example, “longitudinal” or “axial” indicates an axis passing along a center of a catheter from a proximal end to a distal end, or along a center of the catheter handle from a proximal end to a distal end. The term “radial” generally refers to a direction perpendicular to the “axial” direction.

The present disclosure provides a catheter and a large bore introducer suitable for use in the human vasculature for known medical procedures, such as cardiac ablation, electroporation, etc. For purposes of description and explaining the concepts, the present disclosure will be described in connection with a steerable, an introducer, or an introducer handle assembly. It is contemplated, however, that the described features may be incorporated into any number of catheters, introducers, or handles as would be appreciated by one of ordinary skill in the art.

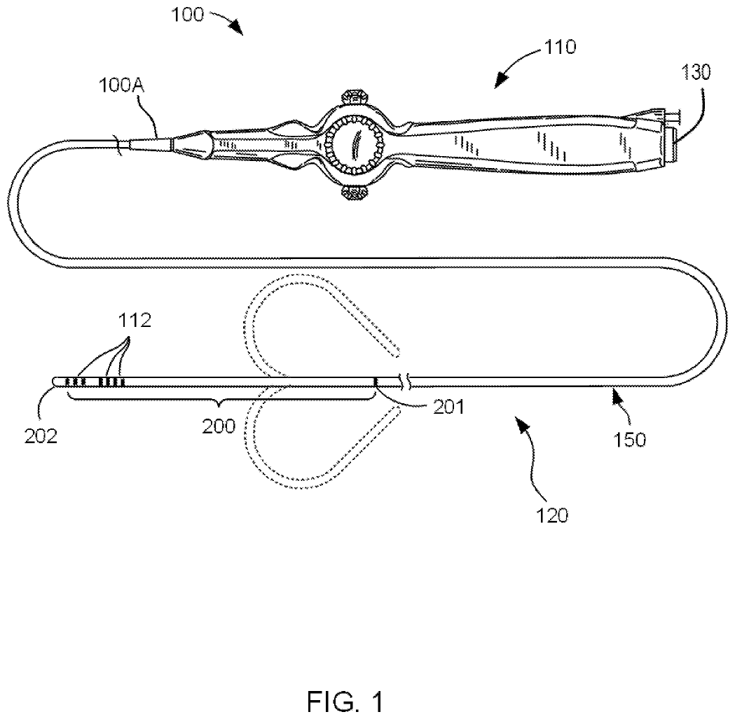

Referring now to the figures, in which like reference numerals refer to the same or similar features in the various views, FIG. 1 shows a steerable catheter 100, in accordance with many embodiments. The steerable catheter 100 includes an elongated catheter shaft assembly 120. The elongated catheter shaft assembly 120 includes a steerable section 200 and a proximal catheter shaft section 150. The steerable section 200 has a distal end 202 and a proximal end 206. The proximal end 206 is coupled with the distal end of the proximal catheter shaft section 150. The steerable section 200 is configured to be selectively curved in either of two directions as illustrated to accommodate navigation of the catheter shaft assembly 120 through a patient's vasculature and/or positioning/orientation of the distal end 202 during a medical procedure. A proximal end 100A of the proximal catheter shaft section 150 is coupled with a handle assembly 110. The handle assembly 110 is configured and operable to selectively curve the steerable section 200.

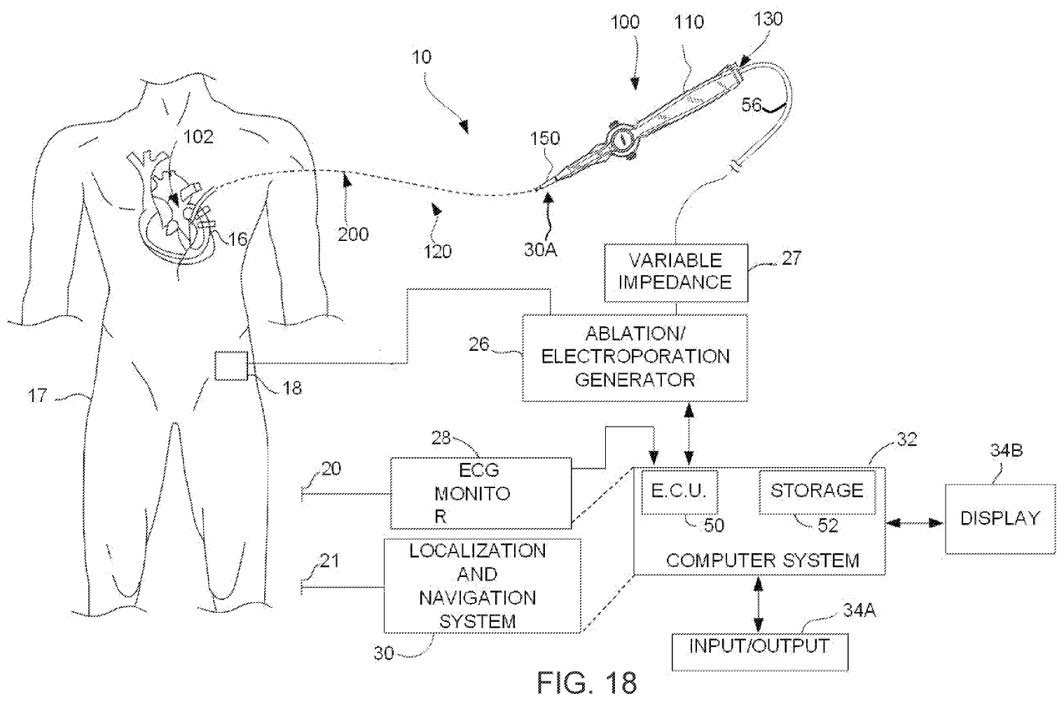

In some embodiments, the steerable catheter 100 includes a diagnostic and/or therapeutic assembly attached to the distal end 202 of the steerable section 200. The diagnostic and/or therapeutic assembly can have any suitable configuration for performing a diagnostic and/or therapeutic medical procedure. For example, in some embodiments, the diagnostic and/or therapeutic assembly includes electrodes 112 configured to accomplish a diagnostic and/or therapeutic medical procedure. For example, the diagnostic and/or therapeutic assembly can include electrodes 112 that are electrically coupled to generator 26 (e.g., as shown in FIG. 18) via suitable electrical wire or other suitable electrical conductors extending through catheter shaft assembly 120). The electrodes 112 may be configured to be selectively energized (e.g., by an electroporation generator 26 and/or computer system 32) to generate a potential and corresponding electric field therebetween, for pulsed field ablation (PFA) therapy. In particular, a system (e.g., in FIG. 18) may be configured for electroporation-induced primary necrosis therapy, which refers to the effects of delivering electric fields in such a manner as to directly cause an irreversible loss of plasma membrane (cell wall) integrity leading to its breakdown and cell apoptosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field (i.e., PFA) in the form of short-duration pulses (e.g., 0.1 to 20 ms duration) between closely spaced electrodes capable of delivering an electric field strength of about 0.1 to 1.0 kv/cm. As described in greater detail below with respect to FIG. 18, the steerable catheter assembly 100 can be used for high output (e.g., high voltage and/or high current) electroporation procedures. The electrodes 112 may be a bipolar electrode assembly, or a monopolar electrode assembly and use a patch electrode (e.g., return electrode 18) as a return or indifferent electrode.

In some embodiments, the steerable catheter 100 is configured as an introducer that includes a lumen configured to accommodate insertion and advancement of a diagnostic and/or therapeutic catheter to a target site within a patient's vasculature. The diagnostic and/or therapeutic catheter can be configured for use in any suitable medical procedure such as, for example, cardiac mapping and/or ablation (e.g., PFA).

The handle assembly 110 is configured to be held by a clinician and operable to articulate the steerable section 200. The handle assembly 110 includes a pull wire actuation mechanism that is drivingly coupled with the steerable section 200 via two pull wires (also referred as deflection wires). The pull wire actuation mechanism includes an input element that is articulable by the clinician to articulate the pull wires to selectively curve the steerable section 200. The handle assembly 110 can be further configured to vary the shape, size, and/or orientation of another portion of the steerable catheter 100 other than the steerable section. The handle assembly 110 can have any suitable configuration, such as configurations that are conventional in the art.

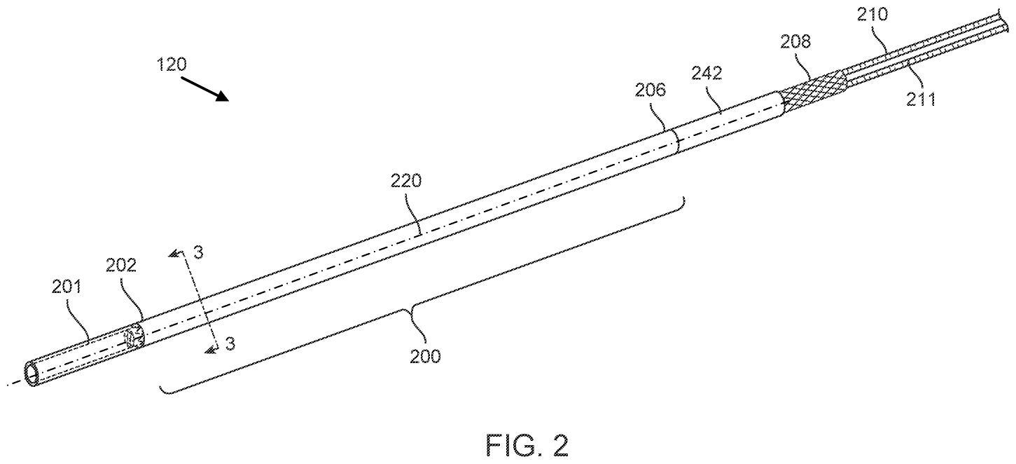

FIG. 2 illustrates an example construction of a distal portion of the elongated catheter shaft assembly 120 that includes the steerable section 200. The steerable section 200 has a centerline 220 (e.g., a longitudinal axis) that extends between the distal end 202 and the proximal end 206. The length and diameter of the steerable section 200 can vary according to the application. For example, the length of the steerable section 200 may range from about 2 inches (18.8 mm) to about 4 inches (101.6 mm) and the diameter of the steerable section 200 may range from about 2 French to about 15 French. In some specific embodiments, the outer diameter of the steerable section 200 can be about 13 French. It should be understood that the dimensions of the steerable section 200 can vary in accordance with various applications of the steerable catheter 100.

In the illustrated embodiment, the catheter shaft assembly 120 includes a distal pocket 201 that is attached to the distal end of the steerable section 200. In many embodiments, the distal pocket 201 is an open lumen that allows for different tip designs to be attached to the catheter shaft. The distal pocket 201 can be made of polymer and adhesive can be used to fill the distal pocket 201 after a tip assembly (not illustrated) is inserted into the open lumen. The adhesive secures the tip assembly to the distal pocket 201 and catheter shaft while providing support for elements (e.g. electrode wires, sensor wires, etc.) routed through the distal pocket 201.

In FIG. 2, portions of the proximal catheter shaft section 150 are not shown to better illustrate different components of the proximal catheter shaft section 150. The proximal catheter shaft section 150 includes pull wire lumens 210, 211 (also referred as deflection lumens). Each of the pull wire lumens 210, 211 is configured to receive a deflection wire and have a suitable flexural stiffness for inclusion in the proximal catheter shaft section 150.

The proximal catheter shaft section 150 can have any suitable configuration. For example, the proximal catheter shaft section 150 can include one or more tubular material layers and one or more tubular braided structures. For example, in the illustrated embodiment, the proximal catheter shaft section 150 includes one or more wires wound to form a tubular braided structure 208 that surrounds the pull wire lumens 210, 211. In the illustrated embodiment, the proximal catheter shaft section 150 includes an outer layer 242 surrounds the tubular braided structure 208. The outer layer 242 can be formed from any suitable material (e.g., a suitable polymeric material such as polyurethane, nylon, or various types of plastic materials such as polyether block amides offered under the trademark PEBAX®, or any other suitable material). The material used to form the outer layer 242 can have the capability to be displaced and/or to shrink when subjected to a process, such as for example, a heating process that is performed during formation of the outer layer 242. The flexibility of the proximal catheter shaft section 150 can be set by setting the flexibility of the tubular braided structure 208 and/or the flexibility of the outer layer 242 via selection of dimensions and material used. Additionally, the flexibility of the proximal catheter shaft section 150, can be varied along the length of the proximal catheter shaft section 150. Alternatively, the flexibility of the proximal catheter shaft section 150 can be substantially constant along the entire length of the proximal catheter shaft section 150.

In the illustrated embodiment, the steerable section 200 is configured to be operable to be selectively curved independent of the proximal catheter shaft section 150. The steerable section 200 can include one or more tubular material layers and one or more tubular braided structures. For example, in the illustrated embodiment, the steerable section 200 includes the tubular braided structure 208 and the outer layer 242. The flexibility of the steerable section 200 can be set by setting the flexibility of the tubular braided structure 208 and/or the flexibility of the outer layer 242 via selection of dimensions and material used. Additionally, the flexibility of the steerable section 200, can be varied along the length of the steerable section 200. Alternatively, the flexibility of the steerable section 200 can be substantially constant along the entire length of the steerable section 200.

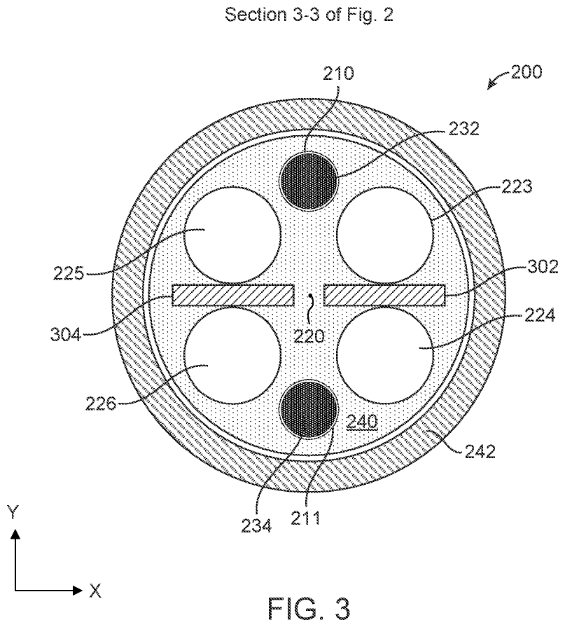

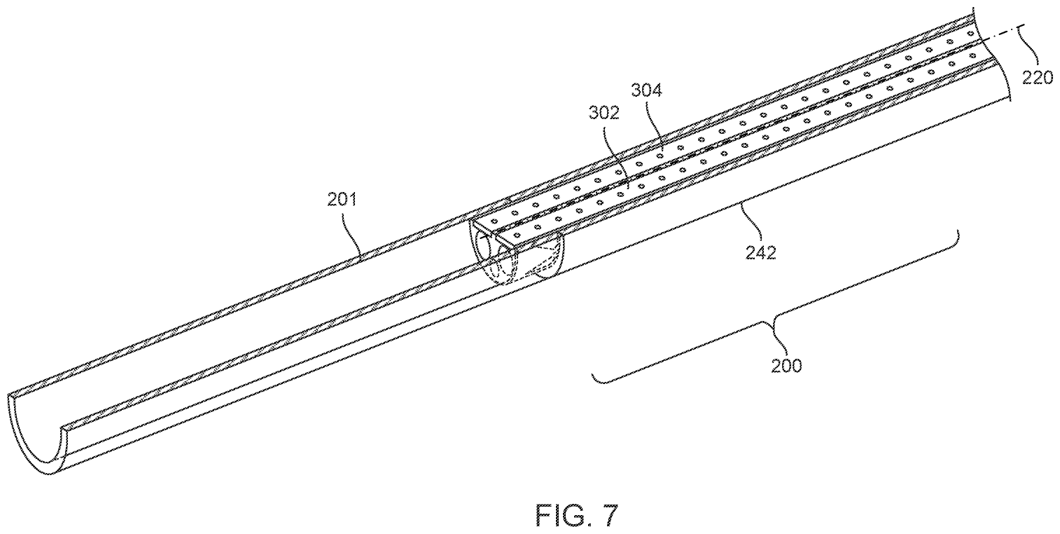

In many embodiments, the steerable section 200 has a minimum cross-sectional bending stiffness in a first direction and a maximum cross-sectional bending stiffness in a second direction that is perpendicular to the first direction to bias deflection of the steerable section 200 in the first direction to enhance planarity of the steerable section 200 during operation of the steerable section 200. In some embodiments, the steerable section 200 includes one or more planarity members (e.g., see 302, 304, 305, 510 in FIGS. 3-6) that increase the differential between the maximum cross-sectional bending stiffness and the minimum cross-sectional bending stiffness. In many embodiments, the one or more planarity members are made from a material with a higher elastic modulus relative to the outer layer 242 so that the one or more planarity members can be sized to leave room within the steerable section 200 for other components (e.g., the pull wire lumens 210, 211, one or more lumens). For example, the one or more planarity members can be formed from a material that has an elastic modulus of at least 20,000 psi. The one or more planarity members are disposed within and attached to the steerable section 200. The one or more planarity members extends along the centerline 220 (e.g., see cross-section view in FIGS. 7 and 8). The one or more planarity members are configured to maximize an amount of cross-section area (e.g., across the diameter) available for lumens used for therapeutic or diagnostic purposes while improving deflection related behavior of the steerable section 200. For example, the one or more planarity members have low torsional stiffness that facilitates improved torsional and flapping behavior of the steerable section 200 experienced during navigating through a tortuous path to reach a target area inside a patient. The one or more planarity members are further discussed in detail below.

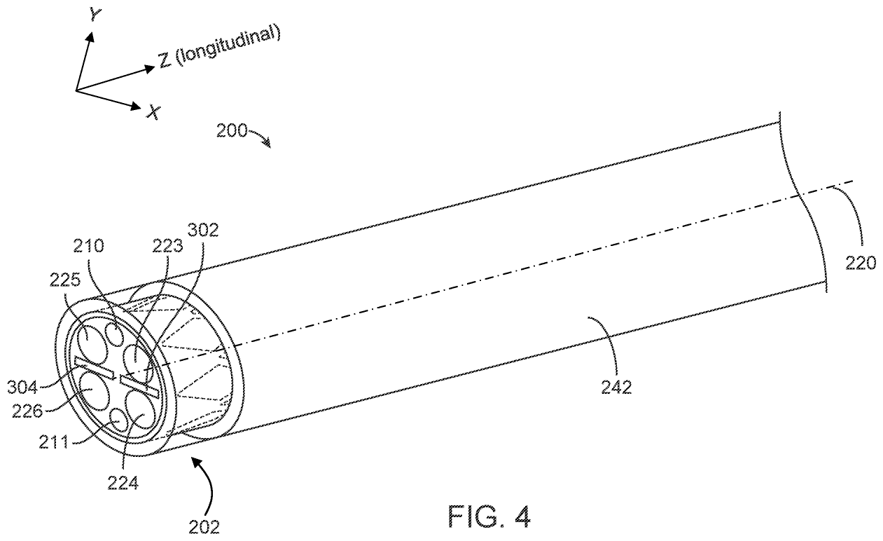

FIG. 3 and FIG. 4 illustrate a cross-section (e.g., along a section line 3-3 in FIG. 2) of the steerable section 200 having two planarity members. In the example of FIG. 3, the steerable section 200 includes a first planarity member 302 and a second planarity member 304. The planarity members are disposed approximately at a center of the cross-section and extend along the centerline 220 (in FIG. 4). The first planarity member 302 is offset from and aligned with the second planarity member 304. The planarity members 302, 304 is attached to the steerable section 200. For example, the planarity members 302, 304 may be glued, molded, or fixedly attached by other means to the steerable section 200. As such, relative motion between the planarity members 302, 304 and the steerable section 200 can be prevented. Hence, when deflecting or undeflecting (e.g., returning to a straight or an initial position prior to deflection), the steerable section 200 and the planarity members 302, 304 move in unison rather than relative to each other thereby providing improved deflection control.

The one or more planarity members (e.g., 302, 304, 305, 510 in FIGS. 3-6) have a cross-sectional area perpendicular to the centerline 220. The cross-sectional area has a minimum principal area moment of inertia and a maximum principal area moment of inertia. In many embodiments, the maximum principal area moment of inertia is at least 5 times the minimum principal area moment of inertia. In some embodiments, the maximum principal area moment of inertia may be at least 15 times, at least 20 times, at least 50 times, at least 100 times, at least 200 times, or other multiples of the minimum principal area moment of inertia. In some embodiments, the minimum principal area moment of inertia is less than 0.0001 inches⁴ (approx. 41.6 mm⁴). In some embodiments, the minimum principal area moment of inertia is less than 0.00001 inches⁴ (approx. 4.16 mm⁴).

The cross-sectional area of the planarity member (e.g., 302, 304, 305, 510) has a width to thickness ratio of at least 5 to 1. In some embodiments, the cross-sectional area of the at least one planarity member has a width to thickness ratio of at least 10 to 1. In many embodiments, the planarity member (e.g., 302, 304, 305, 510) has a substantially rectangular cross-section. For example, the rectangular cross-section may have a width in a range of 0.025 to 0.180 inches and a thickness in a range of 0.002 to 0.006 inches.

Table 1 includes maximum and minimum principal moment of inertial values for some example planarity members. A planarity member with rectangular cross-section has a width (w) and a height (h). The minimum principal moment of inertia of the rectangular cross-section is equal to

$$\frac{1}{12} {bh}^{3} .$$

be computed as to

$$\frac{1}{12} {hb}^{3} .$$

TABLE 1
Principal Inertial Values for Example Planarity Members

| | | (A) Maximum | (B) Minimum | |
|---|---|---|---|---|
| Width | Height | principal area | principal area | Ratio |
| (inches) | (inches) | moment of inertia | moment of inertia | A/B |
| 0.025 | 0.002 | 1.67 × 10⁻¹¹ | 2.60 × 10⁻⁰⁹ | 156 |
| 0.025 | 0.006 | 4.5 × 10⁻¹⁰ | 7.81 × 10⁻⁰⁹ | 17 |
| 0.065 | 0.002 | 4.33 × 10⁻¹¹ | 4.58 × 10⁻⁰⁸ | 1056 |
| 0.065 | 0.006 | 1.17 × 10⁻⁰⁹ | 1.37 × 10⁻⁰⁷ | 117 |

The elongated rectangular shape of the planarity member (e.g., 302, 304, 305, 510) accommodates deflection in one plane while inhibiting deflection in another plane. Additionally, the planarity member(s) have relatively low torsional stiffness due to being thin and not enclosing any area. As a result of the planarity member(s) having relatively low torsional stiffness, the amount of energy stored planarity member(s) due to twisting of the planarity member(s) is relatively low, thereby ensuring that the planarity member(s) does not contribute to any detrimental torsional oscillations of the steerable section 200 during use. A thin rectangular cross-section stores substantial less torsional energy compared to a comparable cylindrical cross-section element. As such, untwisting of the planarity member(s) during operation of the steerable section 200 results in lower detrimental torsional oscillations of the steerable section 200 relative to a comparable cylindrical cross-sectional element.

In the embodiments illustrated in FIG. 3, the steerable section 200 includes the pull wire lumens 210, 211. Each of the pull wire lumens 210, 211 is offset to a respective side from the one or more planarity members 302, 304. The pull wire lumen 210 receives a first pull wire 232 to induce deflection of the steerable section 200 in one direction. The pull wire lumen 211 receives a second pull wire 234 to induce deflection of the steerable section 200 in another direction, opposite to that induced by the first pull wire 232.

The pull wire lumen 211 is disposed on an opposite side (e.g., diametrically opposite) of the one or more planarity members 302, 304 with respect to the pull wire lumen 210. The pull wire lumens 210, 211 are vertically aligned (e.g., along y-axis) approximately along a center of the cross-section of the steerable section 200. In some embodiments, the steerable section 200 is actuatable via articulation of the pull wires 232, 234 to selectively induce curvature in the steerable section 200 down to a radius of curvature of 1 inch.

The planarity member(s) (e.g., 302, 304, 305, 510 having a rectangular cross-section) is thin enough to enable incorporation of one or more lumens of large diameters within the catheter shaft 120. For example, one or more lumens serving different operational, therapeutic or monitoring purposes can be disposed around the planarity members. In some embodiments, additional one or more lumens are incorporated within the steerable section 200 to house and/or convey electrical conductors, fluids, or surgical tools for medical procedures. For example, in the embodiment shown in FIG. 3, the steerable section 200 includes a first lumen 223, a second lumen 224, a third lumen 225, and a fourth lumen 226. The first lumen 223 and the second lumen 224 are disposed on opposite sides of the first planarity member 302. The third lumen 225 and the fourth lumen 226 are disposed on opposite sides of the second planarity member 304. The first lumen 223 and the second lumen 224 may be aligned, while being offset from the centerline 220. Each of the first lumen 223 and the second lumen 224 may have a cross-sectional area that is greater than a cross-sectional area of each of the pull wires lumens 210, 211. Each of the third lumen 225 and the fourth lumen 226 may be offset from the centerline 220 of the steerable section 200. The third lumen 225 may be disposed on the first side of the second planarity member 304, and the fourth lumen 226 may be disposed on the second side of the second planarity member 304. In some embodiments, one or more of the lumens 223-226 may have same diameter. In some embodiments, one or more of the lumens 223-226 may have different diameters. For example, diameters of lumens receiving wires or fluid may be larger than other lumens.

It can be understood that the present example illustrates a quad-lumen configuration without limiting the scope of the present disclosure. In some embodiments, one, two, three, four, five or more lumens may be included. Also, the present disclosure is not limited to a particular distribution of the lumens. In some embodiments, one or more lumens may be aligned with the deflection lumens, horizontally aligned with each other, and/or vertically aligned with each other.

The catheter shaft 120 can include lumen lumens that define the lumens 223, 224, 225, 226. The steerable section 200 includes an inner core 240, which can be formed from a suitable core material. The lumen lumens that define the lumen lumens 223, 224, 225, 226 can be formed from any suitable material, such as the materials described herein that can be used to form the outer layer 242.

The lumens 223-226 can be used to route wires, flexible circuits, or any other element along the length (or even a portion) of the catheter shaft 120. The lumens 223-226 may be used for routing elements from the proximal end of the catheter shaft section 150 to the distal end 202 of the steerable section 200 including, for example, a wire (e.g., a high voltage wire), a vacuum, gas/liquid delivery (e.g., for cryotherapy, medicine, etc.), fiber optic fiber(s), a guide wire, a catheter, an endoscope, optical coherence tomography (OCT) fiber, or other similar devices that a user may need to use. The lumens 223-226 can be separate and independent of each other. For example, the first lumen 223 can be used to transport a fluid and the second lumen 224 can be used to route electrical wires.

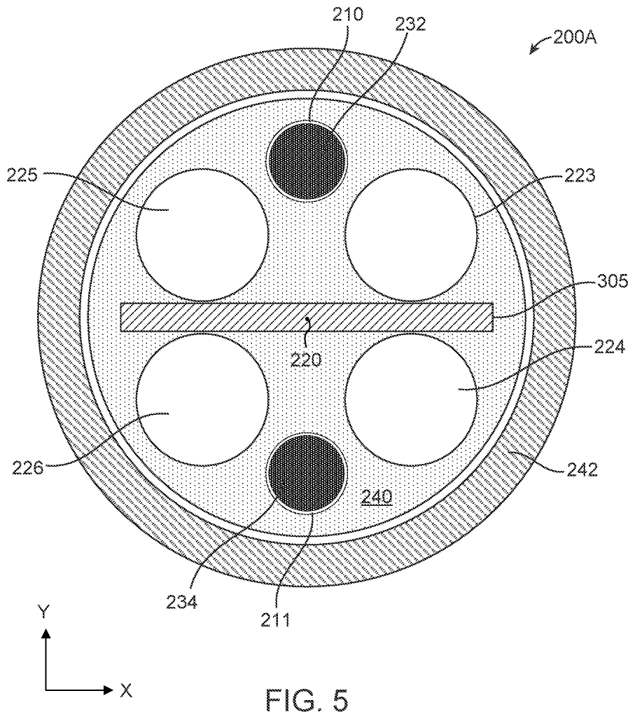

FIG. 5 shows a cross-section (e.g., along the section 3-3 shown in FIG. 2) of an embodiment 200A of the steerable section 200 that includes another example configuration of the planarity member and lumens. As shown, the embodiment 200A includes a single planarity member 305. The single planarity member 305 is disposed approximately at a center of the cross-section and extends along the centerline 220 of the steerable section 200A. The planarity member 305 is fixedly attached to the steerable section 200A via the inner core 240 to prevent relative motion therebetween. The lumen configuration of the steerable section 200A can be the same as or similar to the lumen configuration of the steerable section 200.

In many embodiments, the planarity member 305 has a maximum principal area moment of inertia of at least 20 times the minimum principal area moment of inertia of the planarity member 305. The cross-sectional area of the planarity member 305 may have a width to thickness ratio of at least 5 to 1 or at least 10 to 1. As shown in FIG. 5, the planarity member 305 has a substantially rectangular cross-section. For example, the rectangular cross-section of the planarity member 305 may have a width of about 0.065 inches and a thickness of about 0.002 inches.

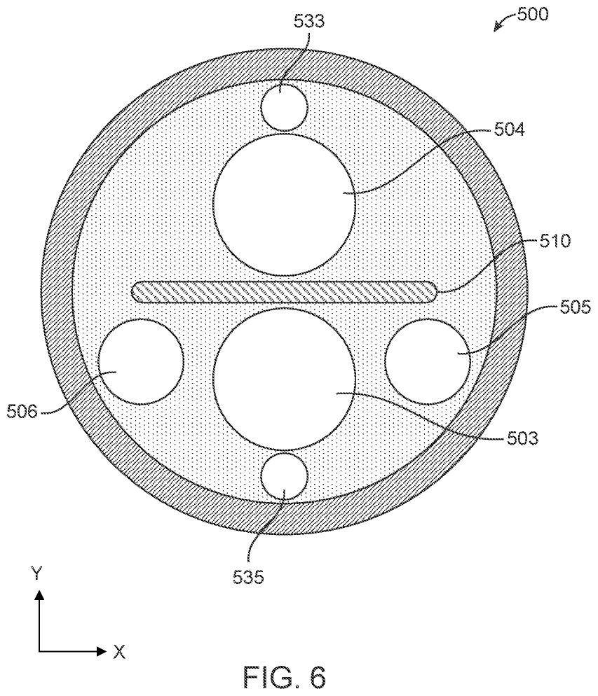

FIG. 6 illustrates a cross-section of an embodiment 500 of the steerable section 200 of an elongated catheter shaft 120 that can be used for electroporation. The embodiment 500 includes a single planarity member 510 disposed approximately at a center of the cross-section and extends along the centerline 220 of the steerable section 200. The planarity member 510 is fixedly attached to the steerable section 200 via the inner core 240 to prevent relative motion therebetween. The single planarity member 510 may have a rounded rectangular cross-section. The cross-sectional area of the planarity member 510 has a width to thickness ratio of at least 5 to 1. The planarity member 510 has have a substantially rectangular cross-section. For example, the rectangular cross-section has a width of about 0.05 inches and a thickness of about 0.002 inches.

The lumen configuration of the embodiment 500 may be different from the cross-sections shown in FIGS. 3 and 5. For example, the distribution of a first pull wire lumen 533, a second pull wire lumen 535, a first lumen 503, and a second lumen 504 around the planarity member 510 is different compared to that shown in FIGS. 3 and 5.

The first lumen 503 and the second lumen 504 is located on opposite sides of the planarity member 510, and vertically aligned with each other. The first lumen 503 and the second lumen 504 are aligned (e.g., along y-axis) with the pull wire lumens 533 and 534 at the center. Additional lumens e.g., a third lumen 505 and the fourth lumen 506 may be included within the steerable section 200. The third lumen 505 and the fourth lumen 506 may be located on the same sides of the planarity member 510, horizontally aligned (e.g., along x-axis) with each other, and offset (e.g., toward left side) from the centerline 220. The first lumen 503 and the second lumen 504 may be of same diameter. The third lumen 505 and the fourth lumen 506 may have same diameter, but smaller than the diameters of the lumens 503 and 504.

The pull wire lumens 533 and 535 carrying the respective pull wire (not shown) may be disposed diametrically opposite to each other on either side of the planarity member 510, similar to that shown in FIGS. 3 and 5. The pull wire lumens 533 and 535 may be vertically aligned (e.g., along y-axis) approximately along a center of the cross-section, placed diametrically opposite to each other on opposite sides of the planarity member 510. The pull wire within the lumens 533 and 535 may induce deflection in a vertical direction (e.g., along y-axis in FIG. 6). In some embodiments, the shape of the planarity member can resist deflection along another axis (x-axis).

As illustrated in FIGS. 3-6, the lumen configuration and the planarity members may be varied, without limiting the scope of the present invention. Depending on the application, type of therapy (e.g., ablation, electroporation, etc.), monitoring capabilities, etc. appropriate lumen configuration (e.g., number of lumens, their locations, sizes, etc.) may be selected. Similarly, depending on the application, type of therapy, and/or desired deflection ability the properties (e.g., number of planarity members, size, shape, material, etc.) of the planarity members may be selected.

FIG. 7 and FIG. 8 show longitudinal cross-sections of a distal portion and a proximal portion, respectively, of the steerable section 200. The cross-sections show two planarity members 302 and 304 attached to the steerable section 200. Each of the planarity members 302 and 304 have a flat plate like structure having flat surfaces with a row of holes. The row of holes serve as attaching means to fixedly attach the planarity members 302, 304 to the steerable section 200. For example, the holes allow the material (e.g., the first material of inner core 240) of the steerable section 200 to pass through during a molding process thereby fusing the planarity members 302 and 304 to the steerable section 200. In some embodiments, the planarity members 304, 304 (and similarly the planarity members 305, 510) have an axial length L1 in a range of 2 inches to 4 inches. In some embodiments, the axial length L1 may extend from the distal end 202 to the proximal end 206 of the steerable section 200.

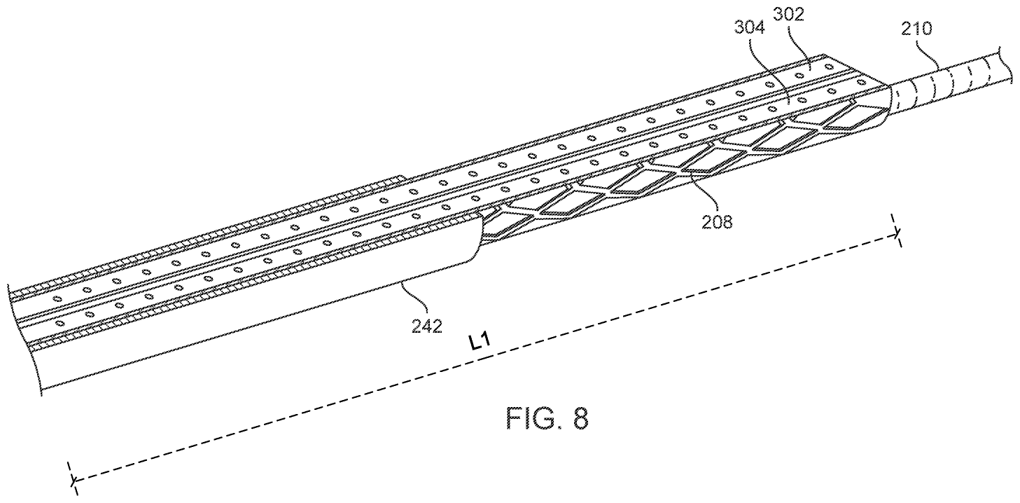

FIG. 9 illustrates an example planarity member 700 with one row of holes 702. The row of holes 702 are formed approximately along a center of the planarity member 700. The holes 702 may be of approximately same size. The holes 702 may be formed equidistant from each other extending along a length of the planarity member 700.

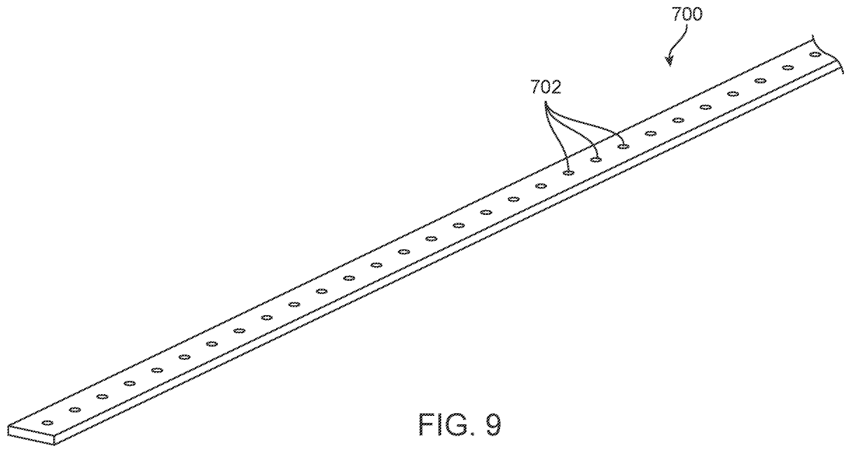

FIG. 10 illustrates another example of a planarity member 710 including a first row of holes 712 and a second row of holes 714. Each row of holes 712, 714 extend along at least partially or along the entire length of the planarity member 710. Each hole may be of approximately same size. The first row of holes 712 may be formed equidistant from each other and offset (e.g., toward left side) from a center of the planarity member 710. Similarly, the second row of holes 714 may be formed equidistant from each other and offset (e.g., toward right side) from a center of the planarity member 710.

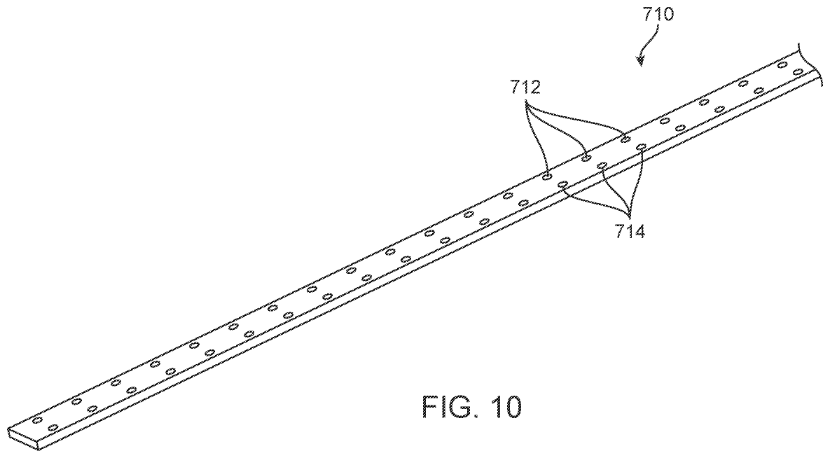

FIG. 11 illustrates yet another example of a planarity member 720 with three rows of holes. The planarity member 720 includes a first row of holes 722, a second row of holes 724, and a third row of holes 726, each row extending along at least partially or along the entire length. Each hole may be of approximately same size. The holes within the row of holes 722, 724, and/or 726 may be formed equidistant from each other. The first row of holes 722 may be offset on one side (e.g., toward left side) of a center of the planarity member 710. The second row of holes 724 may be approximately at a center of the planarity member 720. The third row of holes 726 may be offset on other side (e.g., toward right side) of the center of the planarity member 720.

Any of the planarity members 700, 710, and/or 720 may be used as the planarity members 302, 304, 305, and/or 510, respectively. In some embodiments, same type of planarity member (e.g., 710) may be used as the planarity member 302 and 304. In some embodiments, a combination of planarity members e.g., 710 and 720 may be used as the planarity members 302 and 304, respectively. It can be understood that the planarity members herein are not limited to by a number of holes and/or holes layout. A planarity member with different number of holes, hole sizes, or hole layout (e.g., zigzag, staggered, unevenly distributed, etc.) may be formed. For example, the planarity members 700, 710, and/or 720 may include an opening such as slots, holes of same or different sizes that may or may not be organized in rows. The opening allows material to reflow through the openings and secure the planarity member in-place.

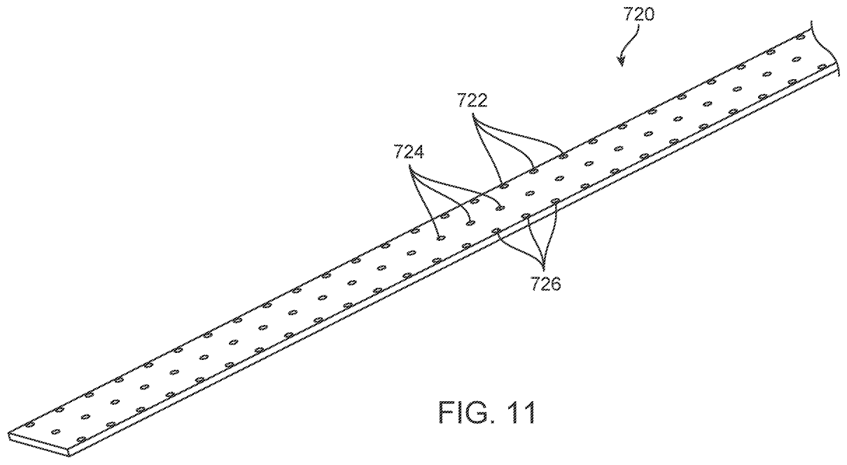

FIGS. 12-14 illustrate different planes with respect to the steerable section 200 and example of deflection along a deflection or bending plane. FIG. 12 shows the catheter shaft 120 in a non-deflected state. The planarity member 305 (or 510) can be seen through the cross-section of the distal end 202 of the steerable section 200. In the example shown, the cross-section of the planarity member 305 is oriented along a particular axis (e.g., y-axis). The steerable section 200 extends, as a substantially tubular structure, between the proximal catheter shaft section 150 and the distal end 202 (e.g., that may include a tip electrode, fluid lumens, etc. in addition to the planarity member 305). In order to deflect the distal end 202, the pull wires 232 and 234 extend from the handle assembly 110 (in FIG. 1) through the steerable section 200 (see also, the cross-section in FIG. 5).

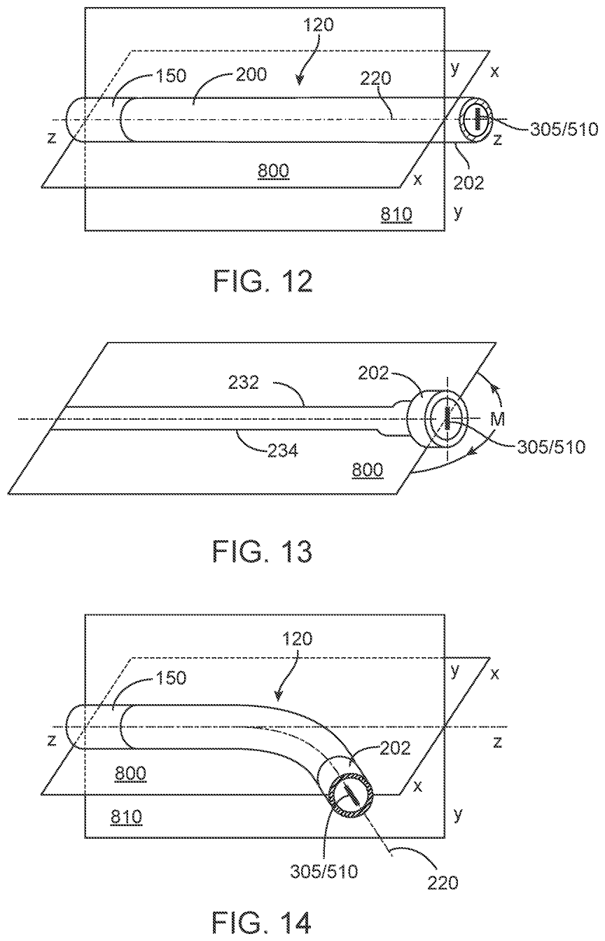

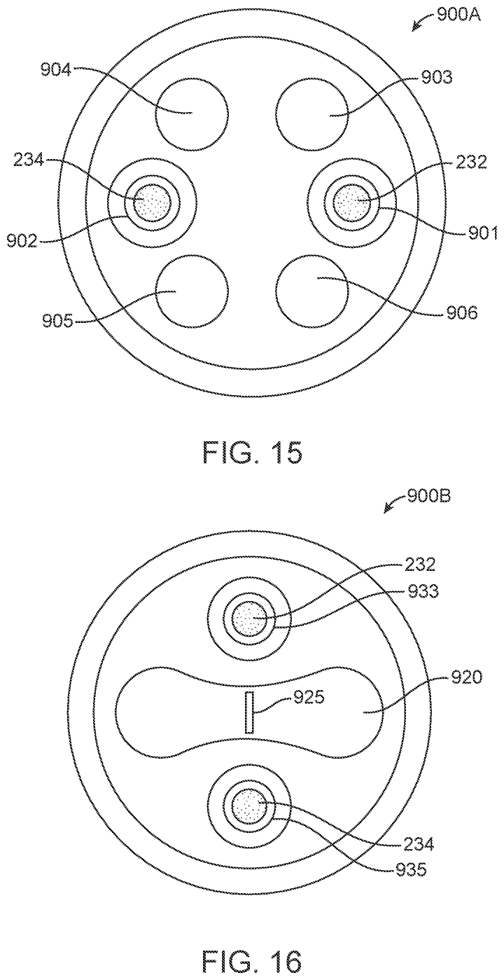

FIG. 13 shows the catheter shaft 120 from FIG. 12 where portions of the elongated catheter shaft section, and a tip (e.g., electrodes and/or lumens) are removed for purposes of illustration. Upon deflection by, for example, manipulating the actuator of the handle (e.g., the handle assembly 110, FIG. 1), the pull wires 232 and 234 generate eccentric pull forces on the distal end 202, which imposes a bending moment M on the steerable section 200. As illustrated in FIG. 14, this deflects a portion of the steerable section 200 and thereby allows for disposing the distal end 202 of the catheter relative to areas of interest inside a patient (e.g., a heart chamber).

Referring to FIG. 14, the distal end 202 (e.g., including electrodes and/or fluid within lumens) of the catheter shaft 120 is caused to move within a bending plane (e.g., deflection plane) 800. As discussed herein, it can be desirable that the deflection of the distal end 202 be constrained only within the bending plane 800. Such constraint to the desired bending plane (i.e., to remain planar, to maintain planarity, etc.) may provide consistent and predictable displacement between deflections of the catheter. The planarity member 305 (or 510), its orientation, and position of the pull wires 232 and 234 can cause the distal end 202 of the catheter shaft 120 to move within the bending plane 800. For example, to ensure the sweeping planarity and consistency of the distal end 202, the planarity member 305 (or 510) may be oriented perpendicular to the bending plane 800, the pull wires 232 and 234 may be placed on either side of the planarity members 305 as shown in FIG. 13 and FIG. 5. As described above, various elements of the catheter can also allow for deflection along the bending plane 800.

The physical configuration of the planarity member 305 (or 700) within the steerable section 200 can be such that it resists deflection in a direction of a plane 810. The plane 810 is perpendicular to the bending plane 800. For example, the flat surfaces of the planarity member 305 (or 510) are positioned within the plane 810, and the pull wires 232 and 234 can be aligned with the bending plane 800 to bend along the bending plane 800 and to resist bending along the plane 810 due to the shape/cross-section of the planarity member 305 (or 510).

Additional information regarding bend planes and planarity of the deflectable section of a distal end of a catheter can be found in U.S. Pat. No. 7,985,215, assigned to St. Jude Medical Atrial Fibrillation Division, filed on Jul. 26, 2011, which is hereby incorporated by reference in its entirety as though fully set forth herein.

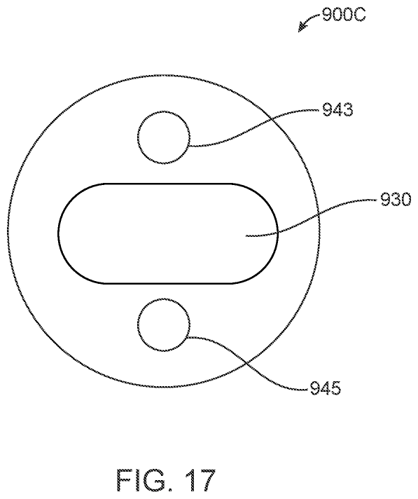

FIG. 15 shows a cross-section 900A that can be employed in a steerable section of the catheter shaft 120, in accordance some embodiments. The cross-section 900A includes pull wire lumens 901 and 902 configured to receive deflection wires 232 and 234, and additional lumens 903-906 configured to facilitate delivery of a therapy to a target portion of a patient, mapping or monitoring of the target portion of the patient. In the present example, planarity members (e.g., 700, 710, 720) may be omitted. In some embodiments, a planarity member may be included at a center of the cross-section 900A.

FIG. 16 shows another example cross-section 900B that can be employed in a steerable section of the catheter shaft 120, in accordance some embodiments. The cross-section 900B includes pull wire lumens 933 and 935 configured to receive deflection wires 232 and 234, and a peanut shaped lumen 920 extending along the centerline 220 (in FIG. 3A). The peanut shaped lumen 920 includes a separator 925 disposed approximately at a center of the peanut shaped lumen 920. The separator 925 divides the lumen 920 into two portions (e.g., a left portion and a right portion) so that each portion may receive a therapy or a monitoring related elements (e.g., electrodes, fluid delivery tubes, etc.). The separator 925 prevents the therapy or monitoring related elements in one portion from crossing or entangling with those in another portion of the lumen 920 during navigating of the catheter shaft 120 to reach a target portion of the patient. In some embodiments, the separator 925 may be a planarity member extending along the centerline 220, as discussed herein. The peanut shaped lumen 920 may be configured to provide planarity of deflection. The peanut shaped lumen 920 may be provided with a liner for maintaining rigidity and planarity, for example as discussed in U.S. patent publication no. 2019/0160256, which is incorporated herein by reference in its entirety.

FIG. 17 shows a cross-section view of another example lumen configuration 900C of a steerable section of the catheter shaft 120, in accordance some embodiments. The lumen configuration 900C includes pull wire lumens 943 and 945 configured to receive deflection wires 232 and 234 (see FIG. 5), and a rounded rectangular shaped lumen 930 disposed approximately at a center of the cross-section and extends along the length of the steerable section 200. The lumen 930 can receive a single planarity member (e.g., similar to 510 in FIG. 6), which may have a rounded rectangular cross-section. In some embodiments, the lumen 930 may receive a therapy or a monitoring related elements (e.g., electrodes, fluid delivery tubes, etc.). The pull wire lumens 943 and 945 are located on opposite sides of the planarity member 510, and vertically aligned with each other.

FIG. 18 is a schematic and block diagram view of a system 10 for electroporation therapy. System 10 can be used for irreversible electroporation (IRE) to destroy tissue.

The system 10 includes a steerable catheter assembly 100 having an elongated catheter shaft 120 coupled to a handle assembly 110. A connector 130 may be coupled at a proximal end of the handle assembly 110 to provide mechanical and electrical connection(s) for cable 56 extending from generator 26. The connector 130 may comprise conventional components known in the art and as shown is disposed at the proximal end of catheter 100. The steerable catheter assembly 100 may also include other conventional components not illustrated herein such as a temperature sensor, additional electrodes, and corresponding conductors or leads disposable via the catheter shaft 120.

A plurality of return electrodes designated 18, 20, and 21, which are diagrammatic of the body connections that may be used by the various sub-systems included in the overall system 1400, such as an electroporation generator 26, an electrophysiology (EP) monitor such as an ECG monitor 28, a localization and navigation system 30 for visualization, mapping and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode. The system 1400 may further include a main computer system 32 (including an electronic control unit 50 and data storage-memory 52), which may be integrated with system 30 provided for visualization, mapping and navigation of internal body structures in certain embodiments. The computer system 32 may further include conventional interface components, such as various user input/output mechanisms 34a and a display 34b, among other components.

The electroporation generator 26 may be configured to energize the electrode element(s) in accordance with an electroporation energization strategy, which may be predetermined or may be user-selectable. A variable impedance 27 allows the impedance of the system to be varied to limit arcing from the catheter electrode of catheter (e.g., 100). Moreover, variable impedance 27 may be used to change one or more characteristics, such as amplitude, duration, pulse shape, and the like, of an output of electroporation generator 26. Although illustrated as a separate component, variable impedance 27 may be incorporated in the catheter 100 or generator 26. In some embodiments, each variable impedance 27 may be connected to a different catheter electrode or group of catheter electrodes to allow the impedance through each catheter electrode or group of catheter electrodes to be separately varied. Additional details of an example electroporation systems are discussed in a PCT publication no. WO2018102376A1, the entire disclosure of which is incorporated herein by reference.

In one or more embodiments of the present disclosure, a steerable catheter assembly includes an elongated catheter shaft section, one or more planarity members, a deflection lumen, and a pull wire. The elongated catheter shaft section having a distal end, a proximal end, and a centerline that extends between the distal end and the proximal end. The one or more planarity members disposed within and attached to the elongated catheter shaft section and extending along the centerline, wherein the one or more planarity members have a cross-sectional area perpendicular to the centerline, wherein the cross-sectional area has a minimum principal area moment of inertia and a maximum principal area moment of inertia, wherein the maximum principal area moment of inertia is at least 5 times the minimum principal area moment of inertia. The deflection lumen offset from the one or more planarity members. The pull wire disposed within the deflection lumen and operable to induce deflection of the elongated catheter shaft section. Optionally, each of the one or more planarity members has a solid cross-sectional area without a closed cell. Optionally, the maximum principal area moment of inertia is at least 50 times the minimum principal area moment of inertia. Optionally, the maximum principal area moment of inertia is at least 100 times the minimum principal area moment of inertia. Optionally, the maximum principal area moment of inertia is at least 200 times the minimum principal area moment of inertia. Optionally, the minimum principal area moment of inertia is less than 0.0001 inches⁴. Optionally, the minimum principal area moment of inertia is less than 0.00001 inches⁴. Optionally, the steerable catheter assembly further includes a second deflection lumen disposed on an opposite side of the one or more planarity members relative to the deflection lumen; and a second pull wire disposed within the second deflection lumen and operable to induce deflection of the elongated catheter shaft section. Optionally, the steerable catheter assembly further includes a third lumen disposed on a first side of the one or more planarity members; and a fourth lumen disposed on a second side of the one or more planarity members opposite to the first side. Each of the third lumen and the fourth lumen has a cross-sectional area that is greater than a cross-sectional area of each of the deflection lumen and the second deflection lumen. Optionally, the steerable catheter assembly further includes a fifth lumen and a sixth lumen, wherein each of the fifth lumen and the sixth lumen is offset from the centerline of the elongated catheter shaft section. Optionally, each of the fifth lumen and the sixth lumen is disposed on the same side of the one or more planarity members and have a cross-sectional area that is smaller than the cross-sectional area of each of the third lumen and the fourth lumen. Optionally, the fifth lumen is disposed on the first side of the one or more planarity members and the sixth lumen is disposed on the second side of the one or more planarity members. Optionally, the one or more planarity members comprises a first planarity member and a second planarity member that is offset from and aligned with the first planarity member. Optionally, each of the one or more planarity members has a substantially rectangular cross-section. Optionally, the one or more planarity members comprise a primary planarity member having a rectangular cross-section with a width in a range of 0.025 to 0.120 inches and a thickness in a range of 0.002 to 0.008 inches. Optionally, each of the one or more planarity members has a length in a range of 1 inches to 5 inches. Optionally, each of the one or more planarity members comprises at least one row of holes extending along a length of a planarity member and fixedly attaching the planarity member to the elongated catheter shaft section. Optionally, each of the one or more planarity members comprises a first row of holes and a second row of holes, wherein each of the first row of holes and the second row of holes extends along the length of the planarity member. Optionally, each of the one or more planarity members is formed from a material having a Young's modulus of at least 20,000 psi. Optionally, the pull wire is operable to induce deflection of the elongated catheter shaft section to produce a radius of curvature of the elongated catheter shaft section of at least 1 inch.

In one or more embodiments of the present disclosure, a bi-directional steerable catheter assembly includes an elongated catheter shaft section, at least one planarity member, a first deflection lumen, a second deflection lumen, a first pull wire, and a second pull wire. The elongated catheter shaft section having a distal end, a proximal end, and a centerline that extends between the distal end and the proximal end. The at least one planarity member disposed within and attached to the elongated catheter shaft section and extending along the centerline, wherein the at least one planarity member has a substantially rectangular cross-sectional area perpendicular to the centerline and at least one row of holes extending along a length of the planarity member and fixedly attaching the planarity member to the elongated catheter shaft section. The first deflection lumen offset from the at least one planarity member. The second deflection lumen offset from the at least one planarity member. The first pull wire disposed within the first deflection lumen and operable to induce deflection of the elongated catheter shaft section in a first direction. The second pull wire disposed within the second deflection lumen and operable to induce deflection of the elongated catheter shaft section in a second direction. Optionally, a material of the at least one planarity member has a higher durometer than a material of the elongated catheter shaft section. Optionally, the at least one planarity member has a width in a range of 0.025 to 0.120 inches and a thickness in a range of 0.002 to 0.008 inches. Optionally, the at least one planarity member has a length in a range of 1 inches to 5 inches. Optionally, the at least one planarity member is a single member having three rows of holes extending along a length of the planarity member. Optionally, the second deflection lumen is disposed on an opposite side of the at least one planarity member relative to the first deflection lumen; and the second pull wire is disposed within the second deflection lumen and operable to induce deflection of the elongated catheter shaft section. Optionally, the bi-directional steerable catheter assembly further includes a third lumen disposed on a first side of the at least one planarity member; and a fourth lumen disposed on a second side of the at least one planarity member opposite to the first side, wherein each of the third lumen and the fourth lumen has a cross-sectional area that is greater than a cross-sectional area of each of the first deflection lumen and the second deflection lumen. Optionally, the bi-directional steerable catheter assembly further includes a fifth lumen and a sixth lumen, wherein each of the fifth lumen and the sixth lumen is offset from the centerline of the elongated catheter shaft section.

In one or more embodiments of the present disclosure, a bi-directional steerable catheter assembly includes an elongated catheter shaft section, at least one planarity member, a first deflection lumen, a second deflection lumen, a first pull wire, and a second pull wire. The elongated catheter shaft section having a distal end, a proximal end, and a centerline that extends between the distal end and the proximal end. The at least one planarity member disposed within and attached to the elongated catheter shaft section and extending along the centerline, wherein the at least one planarity member has a solid cross-sectional area perpendicular to the centerline and without a close cell, wherein the cross-sectional area has a width to thickness ratio of at least 5 to 1. The first deflection lumen offset from the at least one planarity member. The second deflection lumen offset from the at least one planarity member. The first pull wire disposed within the first deflection lumen and operable to induce deflection of the elongated catheter shaft section in a first direction. The second pull wire disposed within the second deflection lumen and operable to induce deflection of the elongated catheter shaft section in a second direction. Optionally, the cross-sectional area of the at least one planarity member has a width to thickness ratio of at least 10 to 1. Optionally, the at least one planarity member has a rectangular cross-section perpendicular to the centerline. Optionally, the at least one planarity member has a width in a range of 0.025 to 0.120 inches and a thickness in a range of 0.002 to 0.008 inches. Optionally, the at least one planarity member has a length in a range of 2 inches to 4 inches. Optionally, the second deflection lumen is disposed on an opposite side of the at least one planarity member relative to the first deflection lumen; and the second pull wire is disposed within the second deflection lumen and operable to induce deflection of the elongated catheter shaft section. Optionally, the bi-directional steerable catheter assembly further includes a third lumen disposed on a first side of the at least one planarity member; and a fourth lumen disposed on a second side of the at least one planarity member opposite to the first side, wherein each of the third lumen and the fourth lumen has a cross-sectional area that is greater than a cross-sectional area of each of the first deflection lumen and the second deflection lumen. Optionally, the bi-directional steerable catheter assembly further includes a fifth lumen and a sixth lumen, wherein each of the fifth lumen and the sixth lumen is offset from the centerline of the elongated catheter shaft section.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A steerable catheter assembly comprising:
an elongated catheter shaft section having a distal end, a proximal end, and a centerline that extends between the distal end and the proximal end;
one or more planarity members disposed within and attached to the elongated catheter shaft section and extending along the centerline, wherein the one or more planarity members have a cross-sectional area perpendicular to the centerline, wherein the cross-sectional area has a minimum principal area moment of inertia and a maximum principal area moment of inertia, wherein the maximum principal area moment of inertia is at least 5 times the minimum principal area moment of inertia;
a deflection lumen offset from the one or more planarity members; and
a pull wire disposed within the deflection lumen and operable to induce deflection of the elongated catheter shaft section.

2. The steerable catheter assembly of claim 1, wherein each of the one or more planarity members has a solid cross-sectional area without a closed cell.

3. The steerable catheter assembly of claim 1, wherein the maximum principal area moment of inertia is at least 50 times the minimum principal area moment of inertia.

4-5. (canceled)

6. The steerable catheter assembly of claim 1, wherein the minimum principal area moment of inertia is less than 0.0001 inches⁴.

7. (canceled)

8. The steerable catheter assembly of claim 1, further comprising:
a second deflection lumen disposed on an opposite side of the one or more planarity members relative to the deflection lumen; and
a second pull wire disposed within the second deflection lumen and operable to induce deflection of the elongated catheter shaft section.

9. The steerable catheter assembly of claim 8, further comprising:
a third lumen disposed on a first side of the one or more planarity members; and
a fourth lumen disposed on a second side of the one or more planarity members opposite to the first side,
wherein each of the third lumen and the fourth lumen has a cross-sectional area that is greater than a cross-sectional area of each of the deflection lumen and the second deflection lumen.

10. The steerable catheter assembly of claim 9, further comprising a fifth lumen and a sixth lumen, wherein each of the fifth lumen and the sixth lumen is offset from the centerline of the elongated catheter shaft section.

11. The steerable catheter assembly of claim 10, wherein each of the fifth lumen and the sixth lumen is disposed on the same side of the one or more planarity members and have a cross-sectional area that is smaller than the cross-sectional area of each of the third lumen and the fourth lumen.

12. The steerable catheter assembly of claim 10, wherein the fifth lumen is disposed on the first side of the one or more planarity members and the sixth lumen is disposed on the second side of the one or more planarity members.

13. The steerable catheter assembly of claim 1, wherein the one or more planarity members comprises a first planarity member and a second planarity member that is offset from and aligned with the first planarity member.

14. The steerable catheter assembly of claim 1, wherein each of the one or more planarity members has a substantially rectangular cross-section.

15. The steerable catheter assembly of claim 14, wherein the one or more planarity members comprise a primary planarity member having a rectangular cross-section with a width in a range of 0.025 to 0.120 inches and a thickness in a range of 0.002 to 0.008 inches.

16. The steerable catheter assembly of claim 1, wherein each of the one or more planarity members has a length in a range of 1 inches to 5 inches.

17. The steerable catheter assembly of claim 1, wherein each of the one or more planarity members comprises at least one row of holes extending along a length of a planarity member and fixedly attaching the planarity member to the elongated catheter shaft section.

18. The steerable catheter assembly of claim 17, wherein each of the one or more planarity members comprises a first row of holes and a second row of holes, wherein each of the first row of holes and the second row of holes extends along the length of the planarity member.

19. The steerable catheter assembly of claim 1, wherein each of the one or more planarity members is formed from a material having a Young's modulus of at least 20,000 psi.

20. (canceled)

21. A bi-directional steerable catheter assembly comprising:
an elongated catheter shaft section having a distal end, a proximal end, and a centerline that extends between the distal end and the proximal end;
at least one planarity member disposed within and attached to the elongated catheter shaft section and extending along the centerline, wherein the at least one planarity member has a substantially rectangular cross-sectional area perpendicular to the centerline and at least one row of holes extending along a length of the planarity member and fixedly attaching the planarity member to the elongated catheter shaft section;
a first deflection lumen offset from the at least one planarity member;
a second deflection lumen offset from the at least one planarity member;
a first pull wire disposed within the first deflection lumen and operable to induce deflection of the elongated catheter shaft section in a first direction; and
a second pull wire disposed within the second deflection lumen and operable to induce deflection of the elongated catheter shaft section in a second direction.

22. The bi-directional steerable catheter assembly of claim 21, wherein a material of the at least one planarity member has a higher durometer than a material of the elongated catheter shaft section.

23.-28. (canceled)

29. A bi-directional steerable catheter assembly comprising:
an elongated catheter shaft section having a distal end, a proximal end, and a centerline that extends between the distal end and the proximal end;
at least one planarity member disposed within and attached to the elongated catheter shaft section and extending along the centerline, wherein the at least one planarity member has a solid cross-sectional area perpendicular to the centerline and without a close cell, wherein the cross-sectional area has a width to thickness ratio of at least 5 to 1;
a first deflection lumen offset from the at least one planarity member;
a second deflection lumen offset from the at least one planarity member;
a first pull wire disposed within the first deflection lumen and operable to induce deflection of the elongated catheter shaft section in a first direction; and
a second pull wire disposed within the second deflection lumen and operable to induce deflection of the elongated catheter shaft section in a second direction.

30. The bi-directional steerable catheter assembly of claim 29, wherein the cross-sectional area of the at least one planarity member has a width to thickness ratio of at least 10 to 1.

31.-36. (canceled)
